(19) United States
(12) Patent Application Publication
(10) Pub. No.: US 2014/0113971 A1
(43) Pub. Date: Apr. 24, 2014

(54) Topical NSAID Compositions Having Sensate Component

(71) Applicants: Joanna ZHANG, West Orange, NJ (US); Emily LESICA, Lebanon, NJ (US)

(72) Inventors: Joanna ZHANG, West Orange, NJ (US); Emily LESICA, Lebanon, NJ (US)

(21) Appl. No.: 14/138,431

(22) Filed: Dec. 23, 2013

Related U.S. Application Data
(63) Continuation of application No. 13/124,292, filed on Jun. 10, 2011.

(51) Int. Cl. A61K 47/10; A61K 47/02; A61K 47/18

(52) U.S. Cl. CPC A61K 47/10; A61K 47/18; A61K 47/02
USPC 514/567

(57) ABSTRACT

Topical pharmaceutical compositions comprising a topically administrable NSAID, a sensate agent and optionally a self-warming system, when administered to a patient in need thereof, provide significant improvements in the rate and extent of skin absorption, as well as impart a sensation of rapid and complete relief from pain.

### BACKGROUND OF THE INVENTION

Various non-steroidal anti-inflammatory drugs (NSAIDs) have been approved for the treatment of pain or inflammation. For example, NSAIDs approved by FDA for the prescription market include naproxen, naproxen sodium, celecoxib, sulindac, oxaprozin, salsalate, piroxicam, indomethacin, etodolac, meloxicam, ketoprofen and nabumetone. NSAIDs approved in the U.S. for the non-prescription market include ibuprofen, naproxen sodium, aspirin, and ketoprofen. All of the foregoing are available in oral dosage forms.

Topical NSAID dosage forms have many advantages for the treatment of arthritis and soft tissue trauma. In particular, they can deliver a high concentration of drug to the desired site of treatment. Currently in the U.S. only diclofenac, as the sodium salt form (i.e. VOLTAREN Gel, 1%), has been approved for topical administration for the relief of pain of osteoarthritis of joints amenable to topical treatment, such as the knees and those of the hands. In Europe and many other countries, diclofenac diethylamine salt, as VOLTAREN Emulgel®, 1.16%, and other NSAIDS in topical dosage forms are available.

### SUMMARY OF THE INVENTION

It has been surprisingly found that the addition of one or more sensate agents and/or a self-warming system to a topical analgesic composition comprising an NSAID dramatically improves the rate and extent of absorption of the drug through mammalian, especially human, skin; and furthermore, that when such compositions are topically applied to the skin of as patient in need thereof, they impart a sensation of rapid and complete relief from pain.

According, the present invention provides topical analgesic compositions to be administered for the treatment of inflammation and pain, and for methods of topically administering said compositions to a patient in need thereof.

In a preferred aspect, the compositions comprise a topically active NSAID; at least one sensate agent; and optionally a “self-warming system” that is capable of transferring heat to the site of administration of the composition, in a topically administrable vehicle.

In a preferred aspect, the sensate agent is a “warming” sensate agent in that it has a warming effect when applied to mammalian, e.g., human, skin.

In one embodiment of the invention, the compositions comprise (1) a topically active NSAID such as diclofenac or a topically administrable salt thereof (e.g., sodium or diethylamine), and (2) at least one warming sensate agent which is a member of the vanilloid family.

For example, the compositions may consist of a topical analgesic composition comprising a topically active NSAID such as diclofenac or salt thereof (e.g., sodium or DEA), and at least one warming sensate agent which is vanillyl butyl ether (VBE).

Alternatively, the compositions may consist of a topical analgesic composition comprising a topically active NSAID such as diclofenac or salt thereof (e.g., sodium or DEA), and at least one warming sensate agent which is capsaicin.

The compositions may additionally comprise a chemical “self-warming system”. By “self warming system” is meant a chemical entity, or a combination of chemical entities, that are capable of generating and transferring heat to the surface to which they are applied, i.e. the skin. In one aspect, the “self-warming system” comprises a chemical entity that produces heat in the presence of a catalyst such as air or the moisture in the skin. In another aspect, the self-warming system may comprise a combination of chemical entities. e.g., an oxidizing agent and a reducing agent (“redox pair”), that are capable of generating an exothermic reaction when combined.

The pharmaceutical compositions according to the invention comprise a therapeutically effective amount of NSAID for the treatment of painful conditions, inflammation and/or rheumatic diseases in warm-blooded animals. The compositions can be administered to treat pain caused by osteoarthritis or rheumatoid arthritis, or muscle pain, joint pain, and back pain. The compositions can be applied, for example, 2 or 3 times, or even 4 times, daily to the intact epidermis. The compositions are particularly useful for the treatment of arthritic pain of the hand or knee by topical application to the afflicted area. Furthermore, the compositions may be used lot the relief of localized pain and inflammation, such as that associated with acute muscular-skeletal injuries.

By “topically active NSAID” is meant an NSAID that is dermatologically acceptable (i.e. skin-tolerable), and that, when administered in a suitable vehicle, can penetrate trans-cutaneously, in particular overcoming the skin barrier of the epidermis, to be locally available at the site of inflammation or injury.

### DETAILED DESCRIPTION

It has been surprisingly found that the addition of one or more sensate agents and/or a self-warming system to a topical analgesic composition comprising an NSAID dramatically improves the rate and extent of absorption of the active agent through mammalian, especially human, skim, and furthermore, when such compositions are topically applied to the skin of a patient in need thereof, they impart a sensation of rapid and complete diminishment of pain.

The NSAID may, for example, be an arylalkanoic acid, such as diclofenac, aceclofenac, acemethacin, a;clofenac, bromfenac, etodolac, indomethacin, nabumetone, oxamethacin, proglumetacin, sulindac, or tolmetin; a 2-arylpropionic acid such as ibuprofen, alminoprofen, carprofen, dexibuprofen, dexketoprofen, fenbufen, fenoprofen, flunoxaprofen, flurbiprofen, ibuproxam, indoprofen, ketoprofen, ketorolac, loxoprofen, naproxen, oxaprozin, pirprofen, suprofen, or tiaprofenic acid; N-arylanthranilic acids, such as mefenamic acid, flufenamic acid, meclofenamic acid, and tolfenamic acid, etc. The term NSAID shall also be inclusive of COX-2 inhibitors such as celecoxib, enolic acid group such as piroxicam and meloxicam, acetylsalicylic acid (i.e. aspirin) and salsalate.

Also included in the term “NSAID” are the pharmaceutically acceptable salts, acids or esters of the foregoing, as well as racemates, enantiomers, and crystal and solvate forms, of the foregoing.

Combinations of the NSAID with effective amounts of one or more other topically active pharmaceutical ingredients, especially analgesic, anesthetic and antipruritic active agents, are also contemplated to be within the scope of the invention. Examples of such active agents include amine and “caine”-type local anesthetics, such as benzocaine, butamben, dibucaine, dibucaine hydrochloride, dimethisoquin hydrochloride, dyclonine hydrochloride, lidocaine, lidocaine hydrochloride, pramoxine hydrochloride, tetracaine, and tetracaine hydrochloride; alcohols and ketones, such as benzyl alcohol, camphor, camphorated metacresol, juniper tar, menthol, phenol, phenolate sodium, and resorcinol; antihistamines, such as diphenhydramine hydrochloride, tripelennamine hydrochloride, hydrocortisone, and hydrocortisone acetate; and counterirritant active, ingredients, such as allyl isothiocyanate, strong ammonia solution, diluted to contain 1 to 2.5% ammonia, methyl salicylate, turpentine oil, menthol, histamine hydrochloride, methyl nicotinate, and capsaicin 0.025 to 0.25 percent.

Suitable topically effective amounts of various NSAIDs, as well as anesthetic and antipruritic active agents, are well-known to the art. For example, diclofenac sodium is approved in U.S. for topical administration for relief of pain of osteoarthritis of the joints in a dosage amount of 20 to 40 mg up to four times daily. The formulated product, Voltaren® Gel, is approved for application to the lower extremities in the amount of 4 grams, 4 times daily, provided that not more than 16 grams be applied daily to any one affected joint of the lower extremities, and for application to the upper extremities in the amount of 2 grams, 4 times daily, provided that not more than 8 grams daily be applied to any one affected joint of the upper extremities; subject to a maximum daily application of 32 grams. Ketoprofen is approved outside the U.S. for the relief of localized pain and inflammation associated with acute muscular-skeletal injuries by topically administering a dosage amount of 100 to 300 mg to the affected area up to two times daily. Other dosage regimens for topical NSAIDs, as well as for the abovementioned analgesic, anesthetic and antipruritic agents, are also known in the art.

The sensate agent preferably directions as a “warming” sensate agent. Physio-logical warming agents include compounds classified as “vanilloids,” i.e. deriving from the vanillyl functional group, which include: vanillyl alcohol alkyl ether derivatives and variations such as vanillyl alcohol n-butyl ether, vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodillydrocapsaicin, and mixtures thereof.

In a preferred embodiment, the warming sensate agent is selected from compounds represented by Formula I and pharmaceutically acceptable salts thereof,

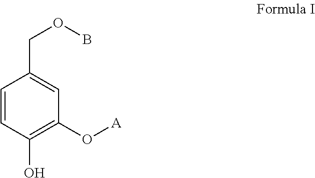

wherein A is an unsubstituted, branched or straight-chained C₁-C₃ alkyl group and B is a hydrogen, or an unsubstituted, branched or straight-chained C₁-C₇alkyl group.

Most preferably in Formula I, A is C₁ alkyl. In a more preferred embodiment, A is C₁ alkyl and B is a C₂-C₄ alkyl. Most preferably, the warming sensate is selected from vanillyl butyl ether (A is a C₁ alkyl and B is a straight-chained, unsubstituted C₄ alkyl) and vanillyl ethyl ether (A is a C₁ alkyl and B is a straight-chained, unsubstituted C₄ alkyl).

In another embodiment the warming sensate is selected from compounds represented by Formula II and pharmaceutically acceptable salts thereof,

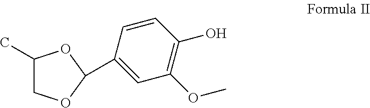

wherein C is an unsubstituted, branched or straight-chained C₂-C₈ alkyl group, optionally interrupted by an oxygen atom.

Thus C may be unsubstituted, straight-chained C₄-C₅alkyl optionally interrupted by an oxygen atom, examples of which are vanillin-1,2-hexylene glycol acetal (C is an unsubstituted, straight-chained C₄ alkyl group) and vanillin-1-butoxyglycerol acetal (C is unsubstituted, straight chained C₅ alkyl interrupted by an oxygen at the 2 position). Warming agents may also be selected from those disclosed in Japanese patent application No. JP 2005-197205, which is incorporated by reference.

Accordingly, suitable agents include vanillyl ethyl ether, vanillyl propyl ether, vanillyl pentyl ether, vanillyl hexyl ether, vanillyl butyl ether acetate, 4(1-menthoxymethyl)-2-phenyl-1,3-dioxolan, 4-(1-menthoxymethyl)-2-(3′,4′-dihydroxyphenyl-1,3-dioxolan, 4(1-menthoxymethyl)-2-(2′-hydroxy-3′-methoxyphenyl)-1,3-dioxolan, 4-(1-menthoxymethyl)-2(4-methoxyphenyl)-1,3-dioxolan, 4(1-menthoxymethyl)-2-(3′,4′-methylenedioxyphenyl)-1,3-dioxolan, 4-(1-menthoxymethyl)-2-(3′-methoxy-4′-hydroxyphenyl)-1,3-dioxolan and analogs (U.S. Pat. Nos. 5,545,424 and 5,753,609, incorporated by reference), nonanoyl vanillyl amide, nonanoic acid vanillyl ether, substituted benzyl alcohol alkyl ether derivatives, vanillin propylene glycol acetal, ethylvanillin propylene glycol acetal, and those warming agents disclosed in U.S. Pat. No. 6,780,443, incorporated by reference.

Other warming sensate agents that mask the sensation of pain include capsicum (red pepper powder, tincture, oil, oleoresin, and extract); ginger extract, oleoresin and oil; Zanthoxylum piperitum extract, sanshool I, sanshool II, sanshoamide, black pepper extract, chavicine, piperine, and spilanthol.

Still other examples of warming sensate agents include ethanol, isopropyl alcohol, iso-amylalcohol, benzyl alcohol, chloroform, eugenol, cinnamon oil, connamic aldehyde and phosphate derivatives of same.

Vanillyl butyl ether (VBE), a preferred warming sensate agent, is commercially available from Takasago, Inc. under the trade name Hotact®, and is typically used in cosmetics for enhancing fragrance (see the International Cosmetic Ingredient Dictionary and Handbook, Monograph ID 12426, published by The Cosmetic, Toiletry, and Fragrance Association (CTFA)).

Also preferred is capsaicin (8-methyl-N-vanillyl-6-nonenamide), another member of the vanilloid family (see CTFA Monograph ID 7655). It is an irritant for mammals, including humans, at certain concentrations producing a sensation of burning in tissue with which it comes into contact. Capsaicin and several related compounds are called capsaicinoids and are derived from chili peppers. Pure capsaicin is a hydrophobic, colorless, odorless, crystalline to waxy compound. When capsaicin is employed as a sensate agent, its concentration should be less than 0.025 wt %.

In a specific embodiment, the composition of the invention comprises the combination of a topically active NSAID such as diclofenac or a topically administrable salt thereof (e.g., sodium or diethylamine), and at least one warming sensate agent which is capsaicin.

In such a composition, the capsaicin is generally present in a concentration of about 0.001 wt. % to about 0.025 wt. %, preferably from about 0.005 to about 0.02 wt. %. Concentrations of capsaicin of 0.025% or greater (e.g., 0.025 wt. % to about 0.25 wt. %) may also be used where it is desirable that capsaicin be included as an active pharmaceutical agent. At such higher concentrations, capsaiein thnctions as an external analgesic as well as a sensate warming agent.

In another embodiment, the invention comprises a topical analgesic composition comprising a topically active NSAID such as diclofenac or a topically administrable salt thereof (e.g., sodium or diethylamine), and at least one warming sensate agent which is VBE.

The VBE is generally included in a concentration of about 0.01 wt. % to about 5 wt. %, preferably 0.1 wt. % to about 3 wt. %.

Optional additional components of the compositions of the invention include one or more agents capable of protecting the composition against UV radiation.

Such agents include inorganic and organic sunscreens that are non-toxic and non-irritating when applied to the skin.

Non-limiting examples of suitable sunscreen agents include, for example, para-aminobenzoic acid (PABA), butyl methoxydibenzoylmethane (avobenzone), benzophenone-1, benzophenone-2, benzophenone-3, benzophenone-4, benzophenone-6, benzophenone-8, benzophenone-12, methoxycinnamate, ethyl dihydroxypropyl-PABA, glyceryl PABA, homosalate, methyl anthranilate, octocrylene, octyl dimethyl PARA, octyl methoxycinnamate, octyl salicylate, 2-phenylbenzimidazole-5 -sulphonic acid, triethanolamine salicylate, 3-(4-methylbenzylidene)-d1-camphor (methylbenzylidene)-bornan-2-one), red petrolatum, 4-Methylbenzylidene-camphor (4-MBC), benzotriazole, phenylbenzimidazole-5-sulfonic acid, methylene bis-benzotrizolyl tetramethylbutyl phenol, diethylamino hydroxybenzoyl hexyl benzoate, and mixtures thereof.

Useful inorganic sunscreens (or sun-blocks) include, but are not limited to, zinc oxide, iron oxide, silica, such as fumed silica, and titanium dioxide. The total amount of inorganic sunscreen that is incorporated in the composition according to the invention is preferably from 0.1 to 3% by weight of the composition.

The preferred sunscreen agents are avobenzone, benzophenone-3, benzophenone-4, octyl methoxycinnamate, diethylamino hydroxybenzoyl hexyl benzoate, zinc oxide, titanium dioxide, and mixtures thereof.

In particular, vanilloid such as VBE is susceptible to taking on a pink coloration when exposed to UV light, and titanium dioxide has been discovered to be effective in stabilizing vanilloid against color change. Microfine titanium dioxide is found to be especially suitable for stabilizing vanilloids against color degradation, and may be in either of its two forms, namely water-dispersible titanium dioxide and oil-dispersible titanium dioxide.

Other optional additional components of the compositions of the invention include additional sensates, especially those that act through the physiological cooling process associated with the TRP melastatin 8 (TRPM8) or cold and menthol receptor 1 (CMR1) channel. Such additional sensates include menthol, as well as menthol derivatives.

In general, examples of cooling sensate agents include compounds represented by Formula III and pharmaceutically acceptable salts thereof,

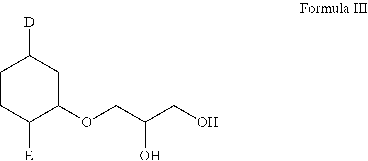

wherein D is a straight chained or branched, unsubstituted C₁-C₄ alkyl or alkenyl group and E is a straight chained or branched, hydroxy-substituted or unsubstituted C₁-C₄ alkyl group.

In a preferred embodiment, the cooling sensate is represented by Formula IV and pharmaceutically acceptable salts thereof,

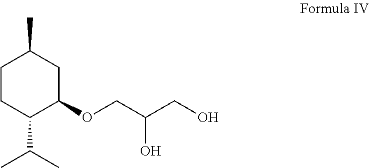 [SMILES: CC(C)[C@@H]1CC[C@@H](C)C[C@H]1OCC(O)CO]

This compound (3-(1)-menthoxypropane-1,2-diol) is commercially available under the name Coolact® 10 and Coolact® P (−)-isopulegol from Takasago Int'l Corp., Tokyo, Japan) and is disclosed in U.S. Pat. No. 4,459,425, incorporated by reference.

Examples of menthol derivatives include menthol carboxamide derivative, cyclohexanecarboxamide, dimethyl menthyl succinimide, menthyl lactate (available under the trade name Frescolat® ML from Symrise GmbH & Co., Holzminden, Germany), menthone glycerin acetal (available under the trade name Frescolat® MGA from Symrise GmbH & Co., Holzminden, Germany), neoisomenthol, neomenthol, isomenthol, PMD 38 p-menthane-3,8,-diol, (2R)-3-(1-menthoxy)propane-1,2-diol, (2RS)-3-(1 -menthoxy)propane-1,2-diol; N-ethyl-5-methyl-2-(1-methylethyl)-cyclohexanecarboxamide (WS-3), ethyleneglycol p-menthane-3carboxylate (WS-4), ethyl 3-(p-menthane-3-carboxamido)acetate (WS-5), N-(4-methoxyphenyl)-p-menthane-3-carboxamide (WS-12), N-t-butyl-p-menthane-carboxamide (WS-14), 2-isopropyl-N-2,3-trimethylbutyramide (WS-23), 1-glyceryl p-menthane-3-carboxylate (WS-30) (all commercially available from Millennium Chemicals, Hunt Valley, Md., USA).

Other cooling sensates which may be included in compositions of the present invention include, but are not limited to, menthol, menthone, camphor, pulegol, isopulegol, cineol, mint oil, peppermint oil, spearmint oil, eucalyptus oil. N-alkyl-p-menthane-3-carboxamide, 3-1-menthoxy-2-methylpropane-1,2-diol, p-menthane-3,8-diol, 2-1-menthoxyethane-1-ol, 1-3-menthoxypropane-1-ol, 4-1-menthoxybutane-1-ol, 1-(2-hydroxy-4,-ethylcyclohexyl)-ethanone, menthyl 3-hydroxybutanoate, menthyl lactate, menthone glycerin ketal, 2-(2-1-menthyloxyethyl)ethanol, menthyl glyoxylate, N-methyl-2,2-isopropylmethyl-3-methylbutanamide, menthyl 2-pyrrolidone-5-carboxylate, monomenthyl succinate, alkali metal salts of monomenthyl succinate, and alkali earth metal salts of monomenthyl succinate, monomenthyl glutarate, alkali metal salts of monomenthyl glutarate, alkali earth metal salts of monomenthyl glutarate, N-[[5-methyl-2-(1-methylethyl)cyclohexyl]carbonyl]glycine, p-menthane-3-carboxylic acid glycerol ester, menthol propylene glycol carbonate; menthol ethylene glycol carbonate, and 6-isopropyl-3,9-dimethyl-1,4-dioxaspiro[4.5]decan-2-one.

Other cooling senates are disclosed in U.S. Pat. Nos. 7,030,273 and 6,780,443, which are incorporated by reference.

The amounts of the above-described second sensate should generally be about 2% or below, e.g., about. 0.5-2 wt. %, e.g. 0.1-1.5 wt. %, of the composition.

In one aspect of the invention, it has been found that ratio of the first “warming” sensate to the second sensate, is desirably about 2:1 to about 1:2 by weight.

In particular, it has surprisingly been discovered that menthol derivatives at low levels (e.g., 0.2 to 2 wt. % of the composition), as an adjunct to VBE in the compositions of the invention, have the salutary effect of reducing skin irritation associated with VBE alone.

Other optional ingredients include skin protectants, such as allantoin, generally at a level of about 0.5% to 2%.

In a further aspect, the invention comprises a topical analgesic composition comprising a topically active NSAID, at least one sensate agent, and furthermore, a self-warming system, in a pharmaceutically acceptable and topically administrable vehicle.

In one embodiment, the self-warming system comprises one or more chemical agents that produce heat in the presence of a catalyst. For example, the self-warming system may include a zeolite that generates heat when the composition comes in contact with the moisture in the skin.

In another embodiment, the self-warming system comprises an oxidizing agent and a reducing agent that generate an exothermic reaction when placed in physical contact with each other.

The compositions of the invention may comprise any delivery vehicle suitable for topical administration to a mammal, such as a suspension, gel, ointment, emulsion, or emulsion gel.

In a preferred aspect, the self-warming system comprises at least one oxidizing agent and at least one reducing agent which are prevented from mutual contacting except at the time of, or just prior to, topical administration of the composition. When said oxidizing agent and reducing, agent (the “redox couple” or “redox pair”) are caused to come in contact with each other, the resulting exothermic reaction produces both an instant and a sustained rise in the temperature of the composition. By “a sustained rise in temperature” is meant an increase of at least about 20° C., above room temperature lasting on the order of at least about 30 seconds, and preferably at least about one minute, after mixing at room temperature.

For example, the composition of the invention may be manufactured and packaged so that it comprises at least two distinct phases: at least a first phase comprising the oxidizing agent(s), and at least as second phase comprising the reducing agent(s). The two phases should be prevented from contacting unless and until the composition is topically administered to a patient in need thereof.

The two or more phases may be manufactured so that they are separated by a physical barrier that is part of the packaging; and the physical barrier is adapted so that it may, by action of the user, be breached to allow mixing together of at least a portion of each of the phases just prior to or during administration.

In still another embodiment, the two or more discrete phases subsist within a single composition of the invention, and means are provided for achieving contacting between the phases upon administration, such as by vigorous mixing or shaking.

For example, a suspension composition may be formed by encapsulating the oxidizing agent in a material which is insoluble in the phase comprising the reducing agent (or vice versa); and by vigorous rubbing of the suspension composition on the patient's skin, the capsules will be disrupted and allow contacting of the redox couple, resulting in heat generation.

If a redox couple is to be included as a “self-warming” component of the composition of the invention, it is usually desirable that the phase of the composition that comprises the reducing agent also contain all of the remaining ingredients except for a portion of the water. However, the phase containing the oxidizing agent may also include, in addition to a portion of the water, any of the remaining ingredients which are inert to the oxidizing agent. For example, for an emulsion gel preparation, a first phase may comprise the reducing agent as well as the NSAID active agent, and the other constituents of the composition; and a discrete second phase may comprise the oxidizing agent in water, with optionally a thickening agent and any other excipient that may be resistant to oxidation.

Suitable oxidizing agents include, but are not limited to, alkali metal salts of perborates, persulfates, carbonate-peroxides and peroxides such as sodium perborate monohydrate, ammonium persulfate, sodium persulfate, potassium persulfate, sodium carbonate peroxide, benzoyl peroxide, calcium peroxide, magnesium peroxide, carbamide peroxide, and hydrogen peroxide. An anhydrous form of hydrogen peroxide is available from International Specialty Products (Wayne, N.J.) in the form of a complex of pharmaceutical grade poly(vinyl pyrrolidone) and hydrogen peroxide. Other suitable peroxides include those summarized in the “Kirk-Othmer Encyclopedia of Chemical Technology”, Fourth Edition, J. I. Kroschwitz and M. Howe-Grant (Editors), Volume 18, pages 202 210 (John Wiley & Sons, 1996). Other oxidizing agents are recited in the International Cosmetic Ingredient Dictionary and Handbook, eds, Wenninger et al., p. 1653 (The Cosmetic, Toiletry, and Fragrance Association, 7th Ed. 1997) (hereinafter the “INCI Handbook”).

Suitable reducing agents include, but are not limited to, thiourea, salts (such as sodium salts) of thiosulfate, sulfite, bisulfite, metabisulfite, borohydride, and hypophosphite, ascorbic acid and salts, esters, and derivatives thereof (e.g., ascorbyl palmitate and ascorbyl polypeptide), and tocopherols and salts, esters, and derivatives thereof (e.g., tocopherol acetate). Other reducing agents are listed on pages 1655-56 of the INCI Handbook.

The amount of oxidizing agent(s) and reducing agent(s) will vary, depending on the size of the substrate, the oxidizing and reducing agents used, and the desired maximum temperature and duration of the exothermic reaction. In one embodiment, the total amount of oxidizing agent(s) and reducing agent(s), independently, is from about 0.005 g to about 0.5 g per square inch of the area to be treated. In one embodiment, the total amount of oxidizing agent(s) and reducing agent(s), independently, is from about 0.01 to about 30%, by weight, of the composition, such as from about 0.1% to about 20% (e.g about 1% to about 10%).

The concentration of oxidizing agents and reducing agents present will depend in part on how much heat is desired and in part on the nature of the by-products which result from the reaction and their effect. It is generally desirable that the total amount of reducing agent be at least as great as the amount required for stoichiometric reaction with all of the oxidizing agents present.

In one embodiment, the equivalent ratio of oxidizing agent(s) to reducing agents(s) in the composition or the article, ranges from about 1:20 to about 20:1, such as from about 1:10 to about 10:1. What is meant by an “equivalent” of an oxidizing or reducing agent is the mass of such substance that will donate or accept one mole of electrons in an oxidation-reduction reaction. For instance, hydrogen peroxide donates two electrons per mole, so its oxidative equivalent is half its molar mass. Sodium sulfite is oxidized by acceptance of two electrons, so its reduction equivalent is half its molar mass. The term “equivalent ratio” refers to the ratio of the equivalents (e.g., of the oxidizing agent(s) to reducing agent(s) in the composition or article), thus factoring in the valency of multi-electron oxidants and reductants for the purposes of outlining desirable excesses of one or the other in practicing this invention.

The target temperature range for the skin-contacting surface of the substrate is between about 30° C., to about 80° C., (e.g., between about 35° C., to 50° C.). In general, if the application duration is short (e.g., less than 10 minutes), the operating temperature may be at the higher end of the above temperature range. However, if the application duration is longer, a lower operating temperature (e.g., less than 42° C., is preferred to avoid heat-related tissue injury for prolonged skin exposure to the composition or article).

In one embodiment, the reducing agent(s) and/or oxidizing agent(s) are in contact with a water-soluble polymer(s). The polymer(s) may be intermixed with or coat the surface of the reducing agent(s) and/or oxidizing agent(s). The presence of the water-soluble polymer may assist in preventing the pre-mature activation of the agents and/or to prevent the agents from directly contacting the skin or eyes of the user. Non-limiting examples of such water-soluble polymer materials include various polyethylene glycols (“PEGs”) such as PEG-32 (Carbowax 1450) and PEG-765 (Carbowax 3350) from Union Carbide (Union Carbide, Midland, Mich.), polyethylene oxides such as PEG-2M (Polyox WSRN-10) and PEG-5M (Polyox WSRN-80) from Amerchol (Edison, N.J.), polyvinyl alcohols such as PVAXX resins C-20 and W-20 (Mitsui Plastics, White Plains, N.Y. USA), cellulose ethers such as hydroxypropyl cellulose, polyvinylpyrrolidone and copolymers of vinyl pyrrolidone such as copolymers of vinyl pyrrolidone and vinyl acetate such as PLASDONE S-630 (ISP, Wayne, N.J., USA), and mixtures thereof.

The weight ratio of water-soluble polymer(s) to the reducing agents(s) and/or oxidizing agents will depend on the type of polymers and agents used and the desired speed of the onset and/or duration of the exothermic reaction. For example, the weight ratio of water-soluble polymer(s) to the reducing agents(s) and/or oxidizing agent(s) can be from about 1:1 to about 100:1, especially from about 2:1 to about 50:1.

An embodiment of the invention utilizes an in-situ formation of sulfate, bisulfate pyrosulfate or mixture thereof from the reaction between sulfites, bisulfites or metabisulfites and peroxide to form sulfates. This embodiment suitably utilizes a 2-phase system that is mixed just prior to topical application. A first phase of the system contains a topically acceptable inorganic cation salt of sulfite, bisulfite metabisulfite or mixture thereof. A second phase contains a topically acceptable peroxide in an amount that exceeds the stoichiometric amount required to convert the sulfite, bisulfite, metabisulfite or mixture thereof, by at least 0.5%. Reaction of the two phases quickly results in a temperature increase of about 3° C., to about 30° C., depending on the concentrations employed.

Ascorbic acid may be used in combination with the sodium sulfite to generate an initial rapid temperature rise followed by a sustained release of heat, as described in U.S. Pat. No. 4,839,081. Additional organic reducing agents which the art teaches can be used instead of or in combination with ascorbic acid include 1.5-diethyl-2-thiobarbituric acid, 2,2′-thiodiethanol; and others. Oxidation of these may be catalyzed with known catalysts such as ammonium molybdate or sodium tungstate.

In one embodiment, to initiate the exothermic reaction where the self-warming system comprises a zeolite, the composition of the invention is wet with water. The water may be added prior to topical application (e.g., wetting the site of topical administration with tap water just prior to use such as less than about five minutes, preferable less than about one minute, prior to use), during application (e.g., applying the composition to water on the skin or providing water from a separate phase of the composition), or after application (e.g., skin perspiration being absorbed into the composition). An example of suitable zeolite is Molsive GMP-4A activated powder (UOP LLC).

The compositions of the present invention can be applied to the skin in two separate steps or simultaneously depending on the type of container used. The two reactive components can be dispensed from physically separate packages or from a unitary package with chambers. Examples of packages include, but are not limited to a pouch inside of a pouch, or a dual bladder system inside of a can, the component of either type of packages can be applied simultaneously or substantially simultaneously to the skin, where they commingle and react. The term “substantially simultaneously” as used herein refers to application of each of the components within temporal proximity to one another not longer than the stability of the initially applied component. Thus there may be two steps to applying the two reactive components: in the first step, one component is applied to the skin and in the second step, the other component is applied over the first component within a period of time less than the stability time of the first component. The components are, thus, applied substantially simultaneously such that commingling occurs when the second component is applied on top of the first component.

In the preparation of the compositions of the invention, the NSAID, sensate agent and optional components of a self-warming system are formulated with a suitable vehicle which is sate for topical use to form, e.g., a solution, a suspension, gel, ointment, emulsion or emulsion gel.

The composition may be anhydrous, such as an anhydrous gel. Such a composition may comprise the pharmaceutically active ingredient, the sensate agent, at least one solvent which is not water, and a thickener, and any other optional ingredients.

Alternatively, the composition may comprise an aqueous gel. Such a composition may comprise the pharmaceutically active agent, the sensate agent, at least one solvent which is water, a thickener, and other optional ingredients.

A still further embodiment comprises an emulsion gel system, comprising the aqueous gel components described above, to which are added an emulsifier, and an emollient or oil.

For example, a topical composition comprising diclofenac may be prepared by dissolving the diclofenac or pharmaceutically acceptable salt thereof in a solvent such as isopropyl alcohol, propylene glycol, or polyethylene glycol. This composition may additionally include water, or may be anhydrous.

A thickener such as a synthetic polymer (e.g., carbomer), or a polysaccharide (e.g., xanthan gum, hydroxypropyl cellulose), may be added to the solvent.

Asehe et al, U.S. Pat. No. 4,917,886, which is hereby incorporated by reference, discloses almost neutral pharmaceutical compositions containing as active ingredient, a non-steroidal, anti-inflammatory compound having at least one acidic group for topical use on intact epidermis, which composition combines within it the properties of a gel with those of an oil/water emulsion

Such compositions have a pH of from approximately 5 to approximately 7.5 and contain from approximately 5 to approximately 50% by weight of a water-soluble, volatile lower alkanol having from 2 up to and including 4 carbon atoms, from approximately 1 to approximately 20% by weight of a co-solvent, from approximately 20 to approximately 80% by weight of water, from approximately 3 to approximately 15% by weight of an optionally self-emulsifying lipid or a mixture of lipids, optionally from approximately 0.5 to approximately 5% by weight of an emulsifier if the lipid phase is not self-emulsifying, from approximately 0.5 to approximately 3% by weight of a gel structure forrner, as active ingredient from approximately 0.1 to approximately 10% by weight of a non-steroidal, anti-inflammatorially active compound, preferably such a compound having at least one acidic group and, if desired, non-essential constituents.

The alcohol component used in the composition according to the invention includes especially lower alkanols having preferably 2 or 3 carbon atoms, such as ethanol or especially isopropanol, and also mixtures thereof. The preferred alcohol proportion in the formulation according to the invention is at least 5% by weight, especially from approximately 10 to approximately 30% by weight.

The function of the co-solvent is to maintain the active ingredient left behind on the skin in solution. In addition, the co-solvent must be miscible with the aqueous-alcoholic phase. Suitable for this purpose are, for example, polyhydric alcohols, such as glycerine, ethylene glycol or propylene glycol, especially poly-lower alkylene glycols, for example polyethylene glycol or polypropylene glycol, having a chain length of from approximately 200 to approximately 6000, preferably from approximately 300 to approximately 1500, units. Preferably, from approximately 5 to approximately 0% by weight are co-solvent.

The fatty phase constituents (including lipids or emollients) that can be used for the novel formulation can be divided into those having non-emulsifying properties and those having self-emulsifying properties. The lipids can be of a vegetable or animal nature and also partly or completely synthetic. Accordingly, there come into consideration as fatty phase constituents, for example, lipids without ester linkages, such as hydrocarbons, fatty alcohols, sterols, fatty acids and salts thereof, and lipids having ester linkages, such as glycerides, waxes and phosphatides. Hydrocarbons having emollient properties include, for example, liquid, semi-solid or solid substances and mixtures, such as paraffins, petroleum jelly, solid paraffin and microcrystalline wax. Fatty alcohols can have, for example, 1 or 2 hydroxy functions and a carbon atom number of approximately from 6 to 34 and be saturated or unsaturated. Those having an even number of carbon atoms, especially those having from 12 to 18 carbon atoms, are preferred. Primary, linear and saturated fatty alcohols are, for example, decanol (capric alcohol), dodecanol (lauryl alcohol), tetradecanol (myristyl alcohol), hexadecanol (cetyl alcohol), octadecanol (stearyl alcohol), cicosanol (arachidyl alcohol), docosanol (behenyl alcohol). The 2-alkyl-fatty alcohols include, for example, 2-hexyl-decanol or 2-octyl-dodecanol. Examples of α-alkanediols that may be mentioned are, for example, 1,12-octadecanediol or 9c-octadecen-1-ol.

Sterols are, for example, naturally tiring steroids that have a 3β hydroxy group and an aliphatic side chain in the 17P-position and are derived, for example, from parent hydrocarbon cholestane, ergostane and stigmastane, such as cholesterol and lanolin.

Fatty acids can be saturated or unsaturated and have, for example, from 6 to 24 carbon atoms, 10 to 18 carbon atoms and an even number of carbon atoms being preferred. Examples of saturated fatty acids are: hexanoic acid (caproic acid), octanoic acid (caprylic acid), decanoic acid (capric acid), dodecanoic acid (lauric acid), tetradecanoic acid (myristic acid), hexadecanoic acid (palmitic acid), octadecanoic acid (stearic acid), eicosanoic acid (arachidic acid), docosanoic acid (behenic acid). Stearic acid is especially preferred. Mono-unsaturated fatty acids are for example: 9-dodecenoic acid (lauroleic acid), 9-tetradecenoic acid (myristoleic acid), 9-hexadecenoic acid (palmitoleic acid), 9-octadecenoic acid (oleic acid), 6-octadecenoic acid (petroselic acid), 9-eicosanoic acid (gadoleic acid), 13-docosenoic acid (erucic acid), while as poly-unsaturated fatty acids there are suitable, for example, 9,12-octadecadienoic acid (linoleic acid) and 9,12,15-oetadecatrienoic acid (linolenic acid). As salts of such fatly acids there come into consideration, for example, alkali metal salts, such as sodium or potassium salts, ammonium salts or amine salts, such as mono-, di- or tri-substituted amines, for example corresponding lower alkylamines or lower alkanolamines, for example corresponding mono-, di- or tri-ethylamines or -ethanol-amines.

Glycerides are intended to mean fatty acid esters of glycerine, it being possible for various fatty acid constituents, for example those mentioned above, to occur within the glyceride. In the case of an increased content of unsaturated fatty acids, the corresponding glycerides are liquid (oils). Glycerides and oils are, for example, groundnut oil (arachis oil), olive oil, castor oil, sesame oil, it being possible also for the oils to be hydrogenated, such as hydrogenated groundnut oil, hydrogenated cotton seed oil, for example Sterotex®, hydrogenated castor oil, for example Cutina® HR. As semi-synthetic and completely synthetic glycerides there come into consideration, for example, caprylic/capric acid triglyceride, for example Miglyol® 812 or Synderm®.GTC, or mono-, di- or tri-esters of palmitic and stearic acid, for example Precirol®.

In order to achieve the desired emollient properties, the fatty phase constituent may also comprise one or more silicone compounds or oils to improve emollient properties on the skin. For example, such silicone compounds may be linear (or straight chain) polymers formed of siloxane bonds, including dimethyl silicone, methylphenyl silicone, and methyl hydrogen silicone fluid. In one embodiment, the dimethyl silicone fluid comprises fully methylated linear siloxane polymers end-blocked with trimethylsiloxy units, i.e., polydimethylsiloxanes, an example of which is Dow Corning Q7-9120 Silicone Fluid, which comprises polydimethylsiloxanes having average kinematic viscosities of from 20 to 12,500 centistokes. Other dimethicones include, for example, Abil 350 (Degussa Care Specialties) and DM Fluids (Shin Etsu).

Other useful silicon compounds include dimethicone copolyols such as dimethicone copolyol and derivatives thereof, such as the acetate, adipate, almondate, amine, butyl ether, laurate, and stearate; as well as dimethicone silylate, dimethicone propylethylenediamine behenate, dimethiconol, octamethyltrisiloxane, polyalkyl siloxane, polyalkylaryl siloxane and alkylmethyl silicone polyglycol.

Still other silicon fluids include cyclic dimethicone or cyclomethicone or cyclopentasiloxane. Suitable cyclic dimethicones include Dow Corning ST-Cyclomethicone 5 NF, SF-1204 (Momentive), and KF9937 and KF9945 (Shin Etsu).

The compositions of the present invention may also contain, from about 0.1% to about 5%, preferably from about 0.5% to about 2%, of a high molecular weight silicone material. This material should be non-polar and should have a molecular weight of at least about 5,000. Examples of such materials are well known in the art and include, for example, polyether siloxane copolymers, crosslinked silicone gels or elastomers, and silicone gums or resins.

In particular, where VBE is employed as the sensate agent, more effective warming is achieved by compositions which are substantially free of hydrocarbon oils. For example, a suitable vehicle for a composition comprising VBE as sensate agent is a silicon emulsion, i.e. wherein one or more silicon fluids comprise the oil component of the emulsion.

Waxes are likewise defined as fatty acid esters but, instead of glycerine, there are suitable as alcohol components alcohols of the sterine series and lower alcohols, for example having from 1 up to and including 12 carbon atoms, such as ethanol, isopropanol or decanol, and also higher even-numbered aliphatic alcohols, for example having from 16 to 36 carbon atoms, especially those mentioned above. Solid and semi-synthetic waxes are, for example, beeswax, carnauba wax, cetyl palmitate, for example Cutina®, wool wax, and lanolin, and liquid waxes are, for example, isopropyl myristate, isopropyl stearate, oleic acid decyl ester, for example Cetiol® V, ethyl oleate, caprylic/capric acid esters of saturated fatty alcohols, especially having from 12 to 18 carbon atoms, for example Cetiol® LC.

As phosphatides there come into consideration especially phosphoglycerides, preferably phosphatidyl cholines which are produced by esterification of sn-glycerine-3-phosphoric acid with a saturated and an unsaturated fatty acid, the phosphoric acid residue being for its part esterified by choline (also called lecithins). For example, egg lecithin or soya lecithin are used.

If, for example, the fatty alcohol is etherified, for example by a lower alkanol or a lower alkoxy-lower alkanol, such as ethanol, a propanol, ethoxyethanol, a methoxy- or ethoxy-propanol, the fatty alcohol may be self-emulsifying, such as ethoxylated fatty alcohols, for example polyhydroxyethylene cetyl stearyl ether, such as Cetomacrogol 1000®.

The fat constituent of the composition according to the invention is preferably from approximately 5 to approximately 10% by weight and can also include mixtures of the compounds mentioned above.

A further constituent of the pharmaceutical preparation according to the invention is emulsifiers the surface-active character of which is determined by the spatially separate lipophilic and hydrophilic centres in the same molecule. Preferably, anion-active surfactants having an acidic hydrophilic group and non-ionogenic surfactants are used.

Corresponding anionic emulsifiers are especially carboxylates, such as readily or sparingly soluble fatty acid salts, salts of fluorinated fatty acids, of alkoxy-carboxylic acids, of sulphonamidocarboxylic acids, of fatty acid lactates, of alkylmalonic or alkylsuccinic acids, sulphonates, for example readily or sparingly soluble alkyl sulphonates, sulphonated fatty acid alkyl esters, fatty acid sulphonates, fatty acid ester sulphonates, perfluorinated alkyl sulphonates, readily or sparingly soluble alkylbenzene sulphonates, and sulphates, for example sulphated primary or secondary fatty alcohols, soaps, esters, amides, alkanolamides, mono- or poly-glycerides, polyglycol ethers, for example of fatty alcohols and alkylphenols. Of the great number of suitable anionic emulsifiers there may be mentioned: soluble soaps, such as sodium palmitate, stearate, oleate and triethanolammonium stearate, alkali metal salts, such as sodium salts, of fatty alcohol sulphates, for example sodium lauryl sulphate or sodium cetyl stearyl sulphate, and sulphosuccinates, such as sodium dioctyl sulphosuccinate.

Non-ionic emulsifiers are, for example, fatty acid esters with mono- or poly-hydric alcohols, such as lower alkanols, ethylene glycol, propylene glycol, with oligohydroxy compounds, such as sorbitol, pentaerythritol or saccharose, or with polyhydroxy compounds, such as polyethylene glycol or polypropylene glycol. Especially suitable are partial glycerine fatty acid esters, glycerine monostearate, partial fatty acid esters of sorbitan, such as sorbitan monolaurate stearate or sesquioleate, partial fatty acid esters of polyhydroxyethylene sorbitan, especially having from approximately 5 to approximately 20 oxyethylene units, such as polyethylene glycol (20)-sorbitan monostearate or monooleate. Other likewise preferred non-ionic emulsifiers are, for example, polyethylene and polypropylene glycol ethers, especially having approximately from 2 to 23 ethylene glycol or ethylene oxide units, of alcohols, such as fatty alcohols, for example of the kind mentioned above, and also polyethers, of fatty acid esters, equally of the etherified and those of the glycerine and sorbitan type, or of fatty amines, such as the corresponding fatty amines derived from the fatty alcohols. Examples of such non-ionic emulsifiers that may be mentioned are: polyhydroxyethylene fatty alcohol ethers, especially having from approximately 12 to approximately 30 mole equivalents of oxyethylene, such as polyhydroxyethylene cetyl stearyl ether, for example Cetomacrogol 1000, polyhydroxyethylene (4)-lauryl ether, polyhydroxyethylene (23)-lauryl ether and others, polyhydroxyethylene fatty acid esters, such as polyhydroxyethylene stearates, especially having from 8 to 1000 oxyethylene groups, for example Myrj 59, and also polyhydroxyethylene glycerine fatty acid esters, for example Tagat S. Also suitable are ethylene oxide and propylene oxide block copolymers having hydrophilic polyhydroxyethylene groups and hydrophobic polyhydroxypropylene groups, for example polyoxyethylenepolyoxypropylene polymers, especially having a molecular weight of from approximately 1000 to approximately 11000, for example Pluronic® F68. Preferred pharmaceutical formulations contain from approximately one to approximately two per cent by weight of emulsifier.

As gel structure formers or viscosity increasing agents in the matrix of which is stored the water necessary for the formulation there are used inorganic and organic macromolecules. The base for high molecular weight inorganic components with gel-forming properties is predominantly water-containing silicates, such as aluminum silicate or magnesium aluminum silicates, such as Veegum®, or colloidal silica, such as Aerosil®. As high molecular weight organic substances there are used, for example, natural, semi-synthetic or synthetic macromolecules. Natural and semi-synthetic polymers are derived, for example, from polysaccharides having the most varied carbohydrate units, such as celluloses, starches, tragacanth, agar-agar, alginic acid and salts thereof, for example sodium alginate, and derivatives thereof, such as lower alkyl celluloses, for example methyl or ethyl celluloses, carboxy- or hydroxy-lower alkyl celluloses, such as carboxymethyl, hydroxyethyl, hydroxypropyl, hydroxypropylmethyl and ethylhydroxyethyl celluloses. Natural and semi-synthetic polymers include, for example, gelatine and gum arabic. The units of synthetic gel-forming macromolecules are, for example, vinyl alcohols, vinyl pyrrolidine, acrylic or methacrylic acid, and as examples of such polymers there may be mentioned polyvinyl alcohol derivatives, especially having a molecular weight of from approximately 28000 to approximately 40000, such as Polyviol® or Moviol®, polyvinyl pyrrolidines, especially having a molecular weight of from approximately 10000 to approximately 1 million, such as Kollidon® or Plasdone®, polyacrylates and polymethacrylates, especially having a molecular weight of from approximately 80000 to approximately I million, or salts thereof, such as Rohagit® S, Eudispert® or carbomer (e.g., Carbopol®). The preferred per cent by weight range when using a gel structure firmer or a mixture thereof is from approximately 0.5 to approximately 3 per cent by weight.

As preferred categories of active ingredient there conic into consideration especially those for systemic treatment that are to be applied to the intact skin, are to enter the skin layers, penetrate these and primarily pass into the circulation of the vascular system of the corium and the subcutis and possibly of the subcutaneous tissue lying beneath the latter and also of the muscle region.

The term “NSAID” shall be understood to include non-steroidal, anti-inflammatorially active compounds having at least one acidic group for systemic treatment, for example, salicylic acid and derivatives thereof, such as acetylsalicylic acid (aspirin), salsalate, diflunisal, flufenamic acid or tolfenamic acid, ketoalkanecarboxylic acids and derivatives thereof, such as fenbufen, aryl- and heteroaryl-alkylcarboxylic acids, such as phenylalkanecarboxylic acids and derivatives thereof, for example diclofenac, ketoprofen, pirprofen, fluoprofen, flurbiprofen, ibuprofen, suprofen, miprofen, and pyrrole-lower alkanecarboxylic acids and derivatives thereof, for example zomepirac, tolmetin or clopirac, lower alkanecarboxylic acids having di- or tri-cyclic aryl and heteroaryl groups, such as naproxen, sulindac, indomethacin, carprofen or pranourofen, also pyrazole compounds, such as pyrazolealkanecarboxylic acids, such as lonazolac or pirazolac, or salts thereof. Especially preferred representatives are, for example, diclofenac and pirprofen and salts thereof.

The preferred proportion NSAID active ingredient is, for example, from approximately 1 to approximately 5% by weight. Salts of active ingredients having acidic groups, such as carboxyl groups, are derived primarily from bases. Corresponding salts are, for example, metal salts, such as alkali metal or alkaline earth metal salts, for example sodium, potassium, magnesium or calcium salts, aluminum salts or transition metal salts, such as zinc or copper salts, or corresponding salts with ammonia or organic amines. Organic amines that come into consideration are, for example, the following: alkylamines, such as mono-, di- or tri-lower alkylamines, alkylenediamines, such as lower alkylenediamines, alkylamines substituted by phenyl, such as mono- or di-phenyl-lower alkylamines, hydroxyalkylamines, such as mono-, di- or tri-hydroxy-lower alkylamines, an oligohydroxy-lower alkylamine or hydroxy-lower alkyl-di-lower alkylamines, amino sugars, for example those in which the amino group can optionally be substituted by at least one lower alkyl radical, cycloalkylamines, such as mono- or di-cyclo-lower alkylamines, basic amino acids, cyclic amines, such as lower alkyleneamines or lower alkenyleneamines having from 2 to -6 carbon atoms, it being possible for the carbon chain also to be interrupted by aza, N-lower alkylaza, oxa and/or thia. Mono-, di- or tri-lower alkylamines are, for example, ethylamine or tert.-butylamine, diethylamine or diisopropylamine, trimethylamine or triethylamine, and lower alkylenediamine is, for example, ethylenediamine. As phenyl-lower alkylamines there come into consideration, for example, benzylamine or 1- or 2-phenylethylamine. Mono-, di- or tri-hydroxy-lower alkylamines are, for example, mono-, di-, tri-ethanolamine or diisopropanolamine; an oligohydroxy-lower alkylamine is, for example, tris-(hydroxymethyl)-methylamine; and hydroxy-lower alkyl-di-lower alkylamines are, for example, N,N-dimethylamino- or from N,N-diethylamino-ethanol. Amino sugars are derived, for example, from monosaccharides in which an alcoholic hydroxy group is replaced by an amino group, such as D-glucosamine, D-galactosamine or marmosamine, N-methyl-D-glucosamine may be mentioned as an example of an N-lower alkylated amino sugar. Mono- or di-cyclo-lower alkylamine is, for example, cyclohexylamine or dicyclohexylamine. Basic amino acids are, for example, arginine, histidine, lysine or ornithine. Lower alkyleneamines and lower alkenyleneamines are, for example, azirine, pyrrolidine, piperidine or pyrroline and as lower alkyleneamines and lower alkenyleneamines of which the carbon chain is interrupted by aza, N-lower alkylaza, oxa and/or thia there are suitable, for example, imidazoline, 3-methylimidazoline, piperazine, 4-methyl- or 4-ethylpiperazine, morpholine or thiomorpholine.

As non-essential constituents of the base substance according to the invention there may be used, if desired, chemical stabilisers. Moisture-retaining agents, if necessary bases for neutralising acidic groups, i.e. groups that yield protons, film formers, perfumes or absorbents.

As chemical stabilisers there come into consideration, for example, anti-oxidants which prevent the oxidative decomposition of active ingredients and adjuncts. Suitable for this purpose are, for example, alkali metal sulphites, such as sodium or potassium sulphite, sodium or potassium bisulphite, alkali metal dithionites, such as sodium or potassium dithionite, or ascorbic acid, and also cysteine, cystine and hydrohalides, such as hydrochlorides. thereof. A preferred stabilizer is sodium sulphite in an amount of about 0.1wt. %. Alternatively, the compositions may be essentially free of stabilizers. Suitable as anti-oxidants for fats, oils and emulsions are, for example, ascorbyl palmitate, tocopherols (vitamin E), phenols, for example propyl gallate, butylhydroxyanisole or butylhydroxytoluene. Additional protection against heavy metal anions, chiefly Cu²⁺ ions, is effected by the addition of complex formers, such as citric acid or, above all, ethylenediaminetetraacetic acid and salts thereof, such as alkali metal or alkaline earth metal salts, for example the corresponding disodium or calcium compounds.

The conditions that must be met by suitable moisture-retaining agents are a high affinity for water, it being necessary that the moisture range be narrow, a high viscosity and good tolerability. In addition, these substances should not have corrosive properties. There come into consideration, above all, polyhydric alcohols having at least two hydroxy functions, such as butanediols, glycerine. sorbitol, mannitol, glucose, ethylene glycol or propylene glycol.

As bases for neutralising acidic groups, i.e. groups yielding protons, there are suitable, for example, those that result in the salts of active ingredients described above. Especially preferred bases are the mentioned organic amines. In addition to the active ingredients, especially gel structure formers having acidic groups are also neutralised,.The addition of base serves especially to adjust the pH value. Consequently, the addition of base may be essential.

Examples of absorbents include micro powders of silica, talc, starch, as well as synthetic polymers such as nylon, etc. and their modified or coated versions. A preferred absorbent in the compositions of the invention is cyclodextrin.

As would be well understood by the worker in the art, the process for preparing the compositions of the invention varies somewhat depending whether the final product is an anhydrous gel or solution, an aqueous gel, or an emulsion gel.

For preparation of an anhydrous gel, the process generally comprises the steps of (1) preparing a solution of the active pharmaceutical agent in a non-aqueous solvent or solvent system; (2) preparing a gel by dissolving in a suitable solvent a gel structure former, optionally with other high molecular weight ingredients such as a film former; (3) combining the solution of (1) and the gel of (2); and (4) adding other ingredients, such as sensate, fragrance etc. Optionally, the selfwarming system is added at this stage. For preparation of an aqueous gel, the process is generally the same as the foregoing, except that at least one of the solvent, used is water, and if the gel structure former has, for example, groups that yield protons, such as carboxy groups, then these groups may. if desired, be neutralized.

For preparation of an emulsion gel according to the invention, the process generally comprises preparing a gel as described above, forming a fatty phase by combining a lipid and/or emollients with heating as necessary; adding the fatty phase to the gel; and, atter cooling to about 40° C. or below, adding the sensate component, and any fragrance or other optional components. Optionally a self-heating agent is also added at this stage. An optional step after combining the fatty phase with the gel is neutralization of the gel structure former and of the active ingredient, if they contain groups that yield protons.

In an alternative procedure, the gel structure former is allowed to swell in a portion of the water, the active ingredient solution is stirred in, neutralized if desired, and then an emulsifier is added to the aqueous phase. Subsequently, the fatty phase and. if desired, the non-essential constituents, are added.

Certain compositions of the invention arc as follows;

A pharmaceutical composition comprising 5-95 wt. % of one or more non-aqueous solvents: 1-4 wt. % ot a topically active non-steroidal anti-inflammatory agent: 0.5-3 wt. % of a sensate agent: and 0.1-3 wt. % of a viscosity increasing agent.

A pharmaceutical composition comprising 15-80 wt. % of one or more non-aqueous solvents: 1-4 wt. % of a topically active non-steroidal anti-inflammatory agent; 0.5-3 wt. % of a sensate agent; 0.5-3 wt. % of a film-former; 0.5-3 wt. % of a gel forming agent; ammonia solution in an amount sufficient to substantially neutralize the pH; optional fragrance; and the remainder water.

A pharmaceutical composition comprising 5-75 wt. % of one or more nonaqueous solvents:. 2-30 wt. % of emolients; 1-10 wt. % of an emulsifier; 1-4 wt .% of a topically active non-steroidal anti-inflammatory agent; 0.5-3 wt. % of a sensate agent; 0.5-3 wt. % of a gel forming agent and/or a viscosity increasing agent; ammonia solution in an amount sufficient to neutralize the pH; 0.1-2 wt. % of fragrance; and the remainder water.

A pharmaceutical composition comprising 15-80 wt. % of one or more non-aqueous solvents; 2-20 wt. % of embollientss; 1-4 wt. % of a topically active non-steroidal anti-inflammatory agent; 0.5-3 wt. % of a sensate agent; 0.5-3 wt. % of a gel forming agent; ammonia solution in an amount sufficient to neutralize the pH; optional fragrance: and the remainder water.

A pharmaceutical composition comprising 5-60 wt. % of one or more non-aqueous solvents; 3-25 wt. % of emollients; 1-10 wt. % of an emulsifier; 1-4 wt. % of a first pharmaceutical agent comprising a topically active non-steroidal anti-inflammatory agent; 1-4 wt. % of a second pharmaceutical agent selected from analgesic, anesthetic and antipruritic active agents; 001-3 wt. % of at least one sensate agent; 0.5-3 wt. % of a gel forming agent; ammonia solution in an amount sufficient to neutralize the pH; optional fragrance; and the remainder water.

A pharmaceutical composition comprising a self-warming system, said composition comprising, two phases,

wherein the first phase comprises 5-40 wt. % of one or more non-aqueous solvents; 1-15 wt. % of emollients; 1-10 wt. % emulsi tie,r; 2-8 wt. % of a topically active non-steroidal anti-inflammatory agent; 0.1-2 wt. % of at least one sensate agent; 0.1-3 wt. % of a viscosity increasing agent; optional fragrance; and the remainder water; and

wherein the second phase comprises 70-90 wt. % non-aqueous solvents; 0.5-10 wt. % of an emollient; 05-10 wt. % of an emulsifier; 1-10 wt. % of self-warming agent; and 0.1-3 wt. % of viscosity increasing agent.

A pharmaceutical composition comprising a self-warming system, said composition comprising two phases,

wherein the first phase comprises 1-40 wt. % non-aqueous solvents; 1-15 wt. % emollients; 1-10 wt. % emulsifier: 0.1-3 wt. % film former: 1-10 wt. % ofa reducing agent; 2-8 wt. % of a topically active non-steroidal anti-inflammatory agent; 0.1-2 wt. % of a sensate agent; 0.1-6 wt. % of viscosity increasing agents; optional fragrance; and the remainder water; and

wherein the second phase comprises .1-65 wt. % non-aqueous solvents; 1-10 wt .% of an oxidizing agent; 0.5-5 wt. % viscosity increasing agent; 0.5-10 wt. % absorbents; and water.

The following Examples illustrate the invention described above but they are not intended to limit the scope thereof in any way. Temperatures are given in degrees Centigrade. Unless otherwise specified, the process is performed at mom temperature (about 22°C.),

### EXAMPLE 1

A composition of the invention is prepared as follows:

| Ingredient | Weight Percent (%) | Function |
|---|---|---|
| Propylene Glycol | 5-50 | Solvent |
| PEG-20 | 5-45 | Solvent |
| Isopropyl Alcohol | 5-20 | Solvent |
| Glycerin | 1-20 | Solvent |
| Diclofenac DEA | 1-4 | Drug substance |
| Vanillyl butyl ether | 0.5-3 | Sensate agent |
| (VBE) | | |
| Hydroxypropyl cellulose | 0.1-3 | Viscosity increasing agent |

Diclofenac DEA is dissolved in a solution of propylene glycol and PEG-20. Hydroxypropyl cellulose is dispersed in isopropyl alcohol and glycerin. The two solutions are combined with mixing to form a Uniform gel. Vanillyl butyl ether is mixd into the gel.

### EXAMPLE 2

A composition in the form of an aqueous gel or solution is prepared as follows:

| Ingredient | Weight Percent (%) | Function |
|---|---|---|
| Propylene glycol | 5-30 | Solvent |
| PEG-8 | 5-30 | Solvent |
| Pentylene glycol | 1-20 | Solvent |
| Isopropyl Alcohol | 5-20 | Solvent |
| Diclofenac sodium | 1-4 | Drug substance |
| VBE | 0.5-3 | Sensate agent |
| Hydroxypropylmethyl | 0.5-3 | Film former |
| cellulose | | |
| Carbomer | 0.5-3 | Gel forming agent |
| Ammonia Solution | 0.1-2.5 | pH adjusting agent |
| (28%) | | |
| Fragrance | 0.1-2 | Fragrance |
| Water | 20-80 | Solvent |

Dielolenac sodium is dissolved in a solution of propylene glycol, pentylene glycol. PEG-8 and a portion of water. Carbomer and hydroxypropylmethyl cellulose are dispersed in isopropyl alcohol and remaining water to form a uniform blend. Ammonia solution is added to the carbomer and cellulose blend and pH is adjusted to the desired range (near neutral). The solution of diclofcriae. DEA is added into the carbomer and cellulose blend and mixed to form a uniform gel. Vanillyl butyl ether and fragrance are added one by one into the gel and mixed to uniformity.

### EXAMPLE 3

An emulsion gel composition is prepared as follows:

| Ingredient | Weight Percent (%) | Function |
|---|---|---|
| PEG-8 | 1-20 | Solvent |
| Propylene glycol | 1-20 | Solvent |
| Isopropyl Alcohol | 5-20 | Solvent |
| Dimethyl isosorbide | 1-15 | Emollient |
| Coco-Caprylate/Caprate | 1-10 | Emollient |
| Petrolatum | 0.1-5 | Emollient/occlusive |
| Dimethicone | 0.1-10 | Emollient/occlusive |
| Polyoxyl 20 | 1-10 | Emulsifier |
| Cetostearylether | | |
| Diclofenac sodium | 1-4 | Drug substance |
| VBE | 0.5-3 | Sensate agent |
| Carbomer | 0.5-3 | Gel forming agent |
| Ammonia Solution | 0.1-2.5 | pH adjusting agent |
| (28%) | | |
| Bisabolol | 0.1-2 | Fragrance |
| Fragrance | 0.1-2 | Fragrance |
| Niacinamide | 0.1-1.25 | Emollient |
| Xanthan gum | 0.1-1 | Viscosity increasing agent |
| Water | 20-80 | Solvent |

Diciorenac sodium is dissolved in a solution of propylene glycol, PEG-8 and a portion of water. Carbomer and xanthan gum are dispersed in isopropyl alcohol, dimethyl isosorbide and remaining water to form an uniform blend. Ammonia solution is added into the carbomer blend and pH is adjusted to the desired range (near neutral). The solution of diclofenac sodium is added into the carbomer blend and mixed to form a uniform gel. The oil phase, consisting of coco-caprylate/caprate, dimethicone, petrolatum and polyoxyl 20 cetostearylether, is formed by melting the constituents together at about 75° C. After the oil phase becomes completely flowable and uniform, it is incorporated into the gel while stirring and mixing, and the product is cooled to about 40° C. Niacinamide is dissolved in a portion of water and added into the product. Fragrance, vanillyl butyl ether and bisabolol are mixed together and added to form the product.

### EXAMPLE 4

A composition according to the invention comprising an emulsion gel with silicone fluids is as follows:

| Ingredient | Weight Percent (%) | Function |
|---|---|---|
| Propylene glycol | 5-30 | Solvent |
| PEG-8 | 5-30 | Solvent |
| Isopropyl alcohol | 5-20 | Solvent |
| Dimethicone | 1-10 | Emollient |
| PEG-12 Dimethicone | 1-10 | Emollient |
| Diclofenac DEA | 1-4 | Drug substance |
| Carbomer | 0.5-3 | Gel forming agent |
| Ammonia Solution | 0.1-2.5 | pH adjusting agent |
| (28%) | | |
| VBE | 0.5-3 | Sensate agent |
| Fragrance | 0.1-2 | Fragrance |
| Water | 20-80 | Solvent |

Diclofenac DEA is dissolved in a solution of propylene glycol, PEG-8 and a portion of water. Carbomer is dispersed in isopropyl alcohol and remaining water to form a uniform blend. Ammonia solution is added into the carbomer blend and pH is adjusted to the desired range (near neutral). The solution of diclolenac DEA is added into the carbomer blend and mixed to form an uniform gel. Dimethicone fluid and PEG- 12 dimethicone are mixed tgother and added into the gel. Vanillyl butyl ether and fragramee are added one by one into the gel and mixed to uniformity.

### EXAMPLE 5

A composition according to the invention comprising an emulsion gel is prepared as follows:

| Ingredient | Weight Percent (%) | Function |
|---|---|---|
| Propylene glycol | 1-20 | Solvent |
| Isopropyl Alcohol | 5-20 | Solvent |
| PEG-8 | 1-20 | Solvent |
| Coco-Caprylate/Caprate | 1-10 | Emollient |
| Mineral oil | 1-10 | Emollient |
| Cetearyl alcohol | 1-5 | Emollient |
| Polysorbate 60 | 1-10 | Emulsifier |
| Diclofenac sodium | 1-4 | Drug substance (NSAID) |
| Lidocaine | 1-4 | Drug substance |
| | | (anesthetic/analgesic) |
| Carbomer | 0.5-3 | Gel forming agent |
| Ammonia Solution | 0.1-2.5 | pH adjusting agent |
| (28%) | | |
| VBE | 0.1-2 | Sensate agent |
| Fragrance | 0.1-2 | Fragrance |
| Capsaicin | 0.01-0.25 | Sensate agent/analgesic |
| Water | 20-80 | Solvent |

Diclofenac sodium and lidocaine are dissolved in a solution of propylene glycol, PEG-8 and a portion of water. Carbomer is dispersed in isopropyl alcohol and remaining water to form an uniform blend. Ammonia solution is added into the carbomer blend and pH is adjusted to the desired range (near neutral). The solution of diclofenac sodium is added into the carbomer blend and mixed to form an uniform gel. The oil phase, consisting of coco-caprylate/caprate, mineral oil, cetearyl alcohol and polysorbate 60, is formed b melting the constituents together at about 75° C., After the oil phase becomes completely flowable and uniform, it is incorporated into the gel while stirring and mixing, and the product is cooled to about 40° C. Fragrance, vanillyl butyl ether, capsaicin and a portion of isopropyl alcohol are mixed together and added to form the product.

### EXAMPLE 6

### Two Phase System (Self-Warming by Hydration of Zeolit)

### Phase A

| Ingredient | Weight Percent (%) | Function |
|---|---|---|
| Propylene glycol | 1-20 | Solvent |
| Isopropyl Alcohol | 5-20 | Solvent |
| Coco-Caprylate/Caprate | 1-10 | Emollient |
| Cetearyl alcohol | 1-5 | Emollient |
| Polysorbate 60 | 1-10 | Emulsifier |
| Diclofenac DEA | 2-8 | Drug substance |
| VBE | 0.1-2 | Sensate agent |
| Xanthan gum | 0.1-3 | Viscosity increasing agent |
| Fragrance | 0.1-2 | Fragrance |
| Water | 20-80 | Solvent |

Diclofenac DEA is dissolved in a solution of propylene glycol and a portion of water. Xanthan gum is dispersed in isopropyl alcohol and remaining water and becomes hydrated to form an uniform gel. The solution of diclofenac DEA is added into the Xanthan gun gel and mixed to uniformity. The oil phase, consisting of coco-caprylate/caprate, cetearyl alcohol and polysorbate 60, is formed by melting the constituents tonther at about 75° C. After the oil phase becomes completely flowable and uniform, it is incorporated into the gel while stirring and mixing, and the product is cooled to about 40° C. Fragrance and vanillyl butyl ether are added one by one into the product.

### Phase B

| Ingredient | Weight Percent (%) | Function |
|---|---|---|
| PEG-8 | 5-45 | Solvent |
| Propylene glycol | 5-50 | Solvent |
| Mineral oil | 0.5-10 | Emollient |
| Glycerin | 1-20 | Solvent |
| Zeolite | 1-10 | Self-warming agent |
| Hydroxypropyl cellulose | 0.1-3 | Viscosity increasing agent |
| Polysorbate 20 | 0.5-10 | Emulsifier |

Hydroxypropyl cellulose is dissolved in a solution of propylene glycol. PEG-8 and glycerin to form a gel matrix. Zeolite is dispersed into the gel matrix. Mineral oil and polysothate 20 are mixed together and added into the product.

Phase A and Phase B are kept from mutual contact until both phases are applied topically to the skin. The self-warming effect is generated while the two phases are mixed together, which is due to the hydration of zeolite in Phase B by water in Phase A and moisture in the skin.

### EXAMPLE 7

### Two Phase. System (Self-Warming by Redox Reaction)

### Phase A

| Ingredient | Weight Percent (%) | Function |
|---|---|---|
| Propylene glycol | 1-20 | Solvent |
| Isopropyl Alcohol | 5-20 | Solvent |
| Coco-Caprylate/Caprate | 1-10 | Emollient |
| Polyoxyl 20 | 1-10 | Emulsifier |
| Cetostearylether | | |
| Petrolatum | 0.5-5 | Emollient/occlusive |
| Polyvinyl alcohol | 0.1-3 | Film former |
| Sodium metabisulfite | 1-10 | Reducing agent/self- |
| | | warming agent |
| Diclofenac sodium | 2-8 | Drug substance |
| Xanthan gum | 0.1-3 | Viscosity increasing agent |
| Sodium polyacrylate | 0.1-3 | Viscosity increasing agent |
| VBE | 0.1-2 | Sensate agent |
| Fragrance | 0.1-2 | Fragrance |
| Water | 20-80 | Solvent |

Diclofenac sodium is dissolved in a solution of propylene glycol and a portion of water. Xanthan gum, sodium polyacrylate and polyvinyl alcohol are dispersed in isopropyl alcohol and a portion of water to form a uniform gel. The solution of diclofenae sodium is added into the Xanthan gum gel and mixed to uniformity. The oil phase, consisting of coco-caprylate/caprate, petrolatum, and polyoxyl 20 cetostearylether, is formed by melting the constituents together at about 75° C. After the oil phase becomes completely flowable and uniform, it is incorporated into the gel while stirring and mixing, and the product is cooled to about 40° C. Sodium inetabisulfite is dissolved in the remaining portion of water and added into the product. Fragrance and vanillyl butyl ether are added one by one into the product.

### Phase B

| Ingredient | Weight Percent (%) | Function |
|---|---|---|
| PEG-8 | 5-45 | Solvent |
| Glycerin | 1-20 | Solvent |
| Hydrogen peroxide | 1-10 | Oxidizing agent/self- |
| | | warming agent |
| Sodium polyacrylate | 0.5-5 | Viscosity increasing agent |
| Talc | 0.5-5 | Absorbent |
| Cyclodextrin | 0.5-5 | Absorbent |
| Water | 20-80 | Solvent |

Sodium polyacrylate is dissolved in a solution of PEG-8, glycerin and water to form a gel matrix. Hydrogen peroxide and ccyclodextrin are mixed together and added into the gel matrix. Talc powders are dispersed into the gel matrix.

Phase A and Phase B are kept from mutual contact until they are applied topically to the skin. The self-warming effect is generated while the two phases are mixed together, which is due to the redox reaction of sodium metabisulfite in Phase A with hydrogen peroxide in Phase B.

### EXAMPLE 8

### Human Skin Permeation Study

A comparison is made of in vitro skin permeation of diclofenac sodium 1% in Voltaren emulgel base (“VEG 1% Na”), with (1) the base plus 1% Hotact® VBE (“VEG 1% Na+1% VBE”) and (2) base plus 0.1% Capsaicin (“VEG 1% Na+0.1% Capsaicin”). The compositions are administered in a single dose at 20 mg/cm², which is equivalent to a daily dose of four applications of 5 mg/cm².

The studies are performed at 35° C., in glass static diffusion Franz cells; approximately 1.75 cm2 area, using human skins. Full-thickness human skin samples from cadaver abdomens are kept frozen at −80°C. until thawed and dermatomed to 0.5 mm, for use. The skin samples are mounted horizontally on the Franz cells, dermis side down. The receptor phase of PBS pH 7.4 (phosphate buffered saline; 7.58 g/L Na2HPO4, 1.62 g/L NaH2PO4 and 4.4 g/L NaCl) contained within each diffusion cell (approximately 8 ml) is mixed using a magnetic stirring.

Permeation of tritiated water is first evaluated to confirm the integrity of skins. After a pre-equilibration period, 400 μl of tritiated water (2.7 μCi/ml) is applied to the surface. After 30 min, the radiolabelled water is removed from the skin with cotton tips. Then 2 ml from the receptor phase is taken in order to measure the amount of tritiated water (%) which permeates across the skin.

20 mg/cm² of the test composition is applied on sample skin having similar tritiated water permeation. Samples of the receptor phase are collected at time intervals: 0, 2, 4, 8, 24 hours, The removed receptor volume (1 ml) is replenished with fresh receptor solution after each withdrawal. The quantities of diclofenao permeating the skin are determined by a HPLC analysis of the collected fractions. A total of 12 different measurements are made per formulation.

In Table 1, the diclofenac permeation rate (μg/cm²) from the VEG 1% Na with 1% VBE is shown to be about 1.4 fold higher than VEG 1% Na within the 24 hour exmitnent duration. A difference is also observed between VEG 1% Na and VEG 1% NA with 0.1%. Capsaicin. Steady-state flux (μg/cm²/h) of diclofenac from all products is reached between 4 and 8 hours.

TABLE 1
Diclofenac permeation expressed as cumulative permeation
(A; μg/cm²) or as flux (B; μg/cm²/hour).

| | | | VEG 1% | | | VEG 1% Na + | | |
|---|---|---|---|---|---|---|---|---|
| | VEG 1% Na | | Na + 1% VBE | | | 0.1% Capsaicin | | |
| Time (hour) | Mean | s.e.m | Mean | s.e.m | Ratio | Mean | s.e.m | Ratio |
| A. Cumulative | | | | | | | | |
| permeation | | | | | | | | |
| (μg/cm²) | | | | | | | | |
| 0 | 0.00 | 0.00 | 0.00 | 0.00 | 0.0 | 0.00 | 0.00 | 0.0 |
| 2 | 0.00 | 0.00 | 0.00 | 0.00 | 0.0 | 0.00 | 0.00 | 0.0 |
| 4 | 0.50 | 0.28 | 0.75 | 0.40 | 1.5 | 0.60 | 0.36 | 1.2 |
| 8 | 1.61 | 0.67 | 2.23 | 0.75 | 1.4 | 1.47 | 0.86 | 0.9 |
| 24 | 7.37 | 2.54 | 10.20 | 3.59 | 1.4 | 8.33 | 2.93 | 1.1 |
| B. Flux | | | | | | | | |
| (μg/cm²/hour) | | | | | | | | |
| 0 | 0.00 | 0.00 | 0.00 | 0.00 | 0.0 | 0.00 | 0.00 | 0.0 |
| 2 | 0.00 | 0.00 | 0.00 | 0.00 | 0.0 | 0.00 | 0.00 | 0.0 |
| 4 | 0.25 | 0.14 | 0.37 | 0.20 | 1.5 | 0.30 | 0.18 | 1.2 |
| 8 | 0.28 | 0.11 | 0.37 | 0.10 | 1.3 | 0.22 | 0.13 | 0.8 |
| 24 | 0.36 | 0.12 | 0.50 | 0.18 | 1.4 | 0.43 | 0.14 | 1.2 |

### EXAMPLE 9

A composition is prepared comprising the composition of Example 2 additionally including:

| | |
|---|---|
| Titanium dioxide | 0.1-3 wt. % (sunblock) |
| Allantoin | 0.1-1 wt. % (anti-irritant) |

### EXAMPLE 10

An emulsion gel composition of the invention is prepared as follows:

| Ingredient | Weight Percent (%) | Function |
|---|---|---|
| Propylene glycol | 1-20 | Solvent |
| Isopropyl Alcohol | 5-20 | Solvent |
| Coco-Caprylate/Caprate | 1-10 | Emollient |
| Polyoxyl 20 | 1-10 | Emulsifier |
| Cetostearylether | | |
| Diclofenac DEA | 1-4 | Drug substance |
| VBE | 0.5-3 | Sensate agent |
| Carbomer | 0.5-3 | Gel forming agent |
| Diethylamine (DEA) | 0.1-2.5 | pH adjusting agent |
| Fragrance | 0.1-2 | Fragrance |
| Xanthan gum | 0.1-1 | Viscosity increasing agent |
| Titanium dioxide | 0.1-3 | Sunblock |
| Water | 20-80 | Solvent |

Diclofentic DEA is dissolved in a solution of propylene glycol and a portion of water. Carbomer and xanthan gum are dispersed in isopropyl alcohol and remaining water to form an uniform blend. DEA is added into the carbomer blend and pH is adjusted to the desired range (near neutral). The solution of diclofenac DEA is added into the carbomer blend and mixed to form a uniform gel. Titanium dioxide is blended with coco-caprylate/caprate and the blend is added into the gel. Polyoxyl 20 cetostearylether is heated at about 75° C.; after it's completely melt, it is incorporated into the gel while stirring and mixing, and the product is cooled to about 40° C. Fragrance and vanillyl butyl ether are added one by one into the product.

| | | |
|---|---|---|
| Propylene glycol | 5-30 | Solvent |
| PEG- 8 | 5-30 | Solvent |
| Isopropyl alcohol | 5-20 | Solvent |
| Dimethicone | 1-5 | Emollient |
| Crosspolymer | | |
| Cyclopentasiloxane | 1-5 | Emollient |
| PEG-12 Dimethicone | 1-10 | Emollient |
| Diclofenac DEA | 1-4 | Drug substance |
| Carbomer | 0.5-3 | Gel forming agent |
| Triethylamine (TEA) | 0.1-2.5 | pH adjusting agent |
| VBE | 0.5-3 | Sensate agent |
| Fragrance | 0.1-2 | Fragrance |
| Water | 20-80 | Solvent |
| Titanium Dioxide | 0.1-3 | Sunblock |
| N-ethyl-5-methyl-2-( 1 | 0.1-2 | Sensate agent |
| methylethyl)- | | |
| cyclohexanecarboxamide | | |
| (WS-3) | | |
| 2-isopropyl-N-2,3- | 0.1-2 | Sensate agent |
| trimethylbutyramide | | |
| (WS-23) | | |

Diclofenac DEA is dissolved in a solution of propylene glycol, PEG-8 and a portion of water. Carbomer is dispersed in isopropyl alcohol and remaining water to form a uniform blend. Triethylamine is added into the carbomer blend and pH is adjusted to the desired range (near neutral). The solution of diclofenac DEA is added into the carbomer blend and mixed to form an uniform gel. Dimethicone crosspolymer, cyclopentasiloxane and PEG- 12 dimethicone are mixed togother with titanium dioxide to form a blend that is added into the gel. Vanillyl butyl ether, fagrance and remaining ingredients are added one by one into the gel and mixed to uniformity

### EXAMPLE 12

### Two Phase System (Self-Warming by Redox Reaction)

### Phase A

| Ingredient | Weight Percent (%) | Function |
|---|---|---|
| Propylene glycol | 1-20 | Solvent |
| Isopropyl Alcohol | 5-20 | Solvent |
| PEG-12 dimethicone | 1-5 | Skin conditioning agent |
| Polyoxyl 20 | 1-5 | Emulsifier |
| Cetostearylether | | |
| Sodium sulfite | 1-10 | Reducing agent/self- |
| | | warming agent |
| Diclofenac diethylamine | 2-8 | Drug substance |
| Xanthan gum | 0.1-3 | Viscosity increasing agent |
| Hydroxyethylcellulose | 0.1-3 | Viscosity increasing agent |
| VBE | 0.1-2 | Sensate agent |
| Fragrance | 0.1-2 | Fragrance |
| Citric acid | 0.5-5 | pH adjusting agent |
| Polyvinylpyrrolidone | 0.1-3 | Film former |
| Water | 20-80 | Solvent |

Diclofenac diethylamine is dissolved in a solution of propylene glycol and a portion of water. Xanthan gum, hydroxyethylcellulose and polyvinyl pyrrolidone are dispersed in isopropyl alcohol and a portion of water to form a uniform gel. The solution of diclofenac DEA is added into the Xanthan gum gel and mixed to uniformity. The oil phase, consisting of PEG-12 dimethicone and polyoxyl 20 cetostearylether, is formed by melting the constituents together at about 75° C. After the oil phase becomes completely flowable and uniform, it is incorporated into the gel while stirring and mixing, and the product is cooled to about 40° C. Sodium sulfite is dissolved in the remaining portion of water and added into the product. Citric acid solution is added to adjust pH. Fragrance and vanillyl butyl ether are added one by one into the product.

### Phase B

| Ingredient | Weight Percent (%) | Function |
|---|---|---|
| Propylene glycol | 1-20 | Solvent/humectant |
| Hydrogen peroxide | 1-10 | Self-warming reagent |
| Coco-Caprylate/Caprate | 1-5 | Emollient |
| PEG-100 stearate | 1-5 | Emulsifier |
| Cetyl alcohol | 1-5 | Emollient/Co-emulsifier |
| Stearic acid | 1-5 | Emollient/Co-emulsifier |
| Triethyl amine (TEA) | 0.1-2 | pH adjusting agent |
| Water | 20-80 | Solvent |

Phase A and Phase B are kept from mutual contact until they are applied topically to the skin. The self-warming effect is generated while the two phases are mixed together, which is due to the redox reaction of sodium sulfite in Phase A with hydrogen peroxide in Phase B.

We claim:

1. A topical pharmaceutical composition for the relief of pain or inflanunation in a patient in need thereof comprising a topically active non-steroidal anti-inflammatory agent and at least one sensate agent, and optionally a self-warming system, in a topically administrable vehicle.

2. A composition according to claim 1 wherein the topically active non-steroidal anti-inflammatory agent comprises diclofenac or a pharmaceutically acceptable salt tliereof.

3. A composition according to claim 1 wherein the at least one sensate agent comprises a physiological warming agent.

4. A composition according to claim 3 wherein the at least one sensate agent comprises a vanilloid.

5. A composition according to claim 4 wherein the vanilloid is selected from vanilyl alcohol n-butyl ether, vanillyl alcohol n-propyl ether, vanillyl alcohol isopropyl ether, vanillyl alcohol isobutyl ether, vanillyl alcohol n-amino ether, vanillyl alcohol isoamyl ether, vanillyl alcohol n-hexyl ether, vanillyl alcohol methyl ether, vanillyl alcohol ethyl ether, gingerol, shogaol, paradol, zingerone, capsaicin, dihydrocapsaicin, nordihydrocapsaicin, homocapsaicin, homodihydrocapsaicin, and mixtures thereof.

6. A composition according to according to claim 3 wherein the physiological warming agent comprises vanillyl vanillyl butyl ether,

7. A composition according to claim 3 wherein the physiological warming agent comprises capsaicin.

8. A composition according to claim 5 wherein the topically active non-steroidal anti-inflammatory agent comprises diclofenac or a pharmaceutically acceptable salt thereof.

9. A composition according to claim 7 wherein the topically active non-steroidal anti-inflammatory agent comprises diclofenac or a pharmaceutically acceptable salt thereof.

10. A composition according to claim 5 which comprises one or more additional sensate agents that act via the physiological cooling process associated with the TRP melastin 8 (TRPM8) or cold and menthol receptor 1 (CMR1) channel.

11. A composition according to claim 10 wherein the one or more additional sensate agents are selected from menthol and menthol derivatives, and mixtures thereof.

12. A composition according to claim 11 wherein the one or more additional sensate agents are selected from N-ethyl-5-methyl-2-(1-methylethyl)-cyclohexanecarboxamide, 2-isopropyl-N-2,3-trimethyl- butyramide, and mixtures thereof.

13. A composition according to claim 6 which additionally comprises an agent capable of protecting the composition against UV radiation.

14. A composition according to claim 13 wherein the agent is titanium dioxide.

15. A composition according to claim 3 wherein the topically active non-steroidal anti-inflammatory agent comprises diclofenac or a pharmaceutically acceptable salt thereof, the physiological warming agent comprises a anilloid or a mixture thereof; and the optional self-warming system comprises a reducing agent and an oxidizing agent.

16. A composition according to claim 15 wherein the reducing agent comprises a thiosulfate, sulfite, bisulfite, or metabisulfite, or a salt thereof, and the oxidizing agent comprises a peroxide.

17. A composition according to claim 16 wherein the reducing agent and the oxidizing agent are prevented from mutual contacting until the composition is administered, whereupon the resulting exothermic reaction produces an instant and sustained rise in the temperature of the composition.

18. A composition according to claim 3 wherein the topically active non-steroidal anti-inflammatory agent comprises diclofenac or a pharmaceutically acceptable salt thereof; and the at least one physiologically warming agent comprises vanillyl butyl ether.

19. A composition according to claim 18 which comprises one or more additional sensate agents that act via the physiological cooling process associated with the TRP melastatin 8 (TRPM8) or cold and menthol receptor 1 (CMR1) channel.

20. A composition according to claim 19 wherein the one or more additional sensate agents are selected from menthol and menthol derivatives, and mixtures thereof.

21. A composition according to claim 20 wherein the one or more additional sensate agents comprise about 0.2 to 2 wt. % of the composition.

22. A composition according to claim 20 wherein the one or more additional sensate agents are selected from N-ethyl-5-methyl-2-(1-methylethl)-cyclohexanecarboxamide, 2-isopropyl- N-2,3-trimethyl- butyramide, arid mixtures thereof.

23. A composition according to claim 18 which additionally comprises an agent capable of protecting the composition against UV radiation.

24. A composition according to claim 23 wherein the agent is titanium dioxide.

25. A composition according to claim 1 wherein, the topically administrable vehicle comprises an emulsion gel.

26. A topically administrable pharmaceutical composition for the relief of pain or inflammation in a patient in need thereof comprising a topically active non-steroidal anti-inflammatory agent and at least one sensate agent, and optionally a self-warming system, in a topically administrable vehicle comprising:
(a) from approximately 5 to approximately 50% by weight of a water-soluble, volatile lower alkanol having from 2 up to and including 4 carbon atoms,
(b) from approximately 1 to approximately 20% by weight of a polyhydric alcohol or a poly-lower alkylene glycol having a chain length of from approximately 200 to approximately 6000 units as co-solvent,
(c) from approximately 20 to approximately 80% by weight of water,
(d) from approximately 3 to approximately 15% by weight of a liquid, semi-solid or solid hydrocarbon: a fatty alcohol having 1 or 2 hydroxy functions and approximately from 6 to 34 carbon atoms; a fatty acid ester with glycerine, the fatty acid having from 6 to 24 carbon atoms; a fatty acid ester of a lower alcohol, having from 1 up to and including 12 carbon atoms or of a higher even-numbered aliphatic alcohol having from 16 to 36 carbon atoms, the fatty acid having from 6 to 34 carbon atoms; or a fatty alcohol of approximately from 6 to 34 carbon atoms etherified it lower alkanol or a lower alkoxy-lower alkanol; as a lipid; or a silicon compound selected from dimethyl silicone, methylphenyl silicone, methyl hydrogen silicone, fully methylated linear siloxane polymers end-blocked with trimethylsiloxy units, polydimethylsiloxanes, dimethicone copolvols, dimethicone copolbvol and the acetate
(e) in the presence or absence of from approximately 0.5 to approximately 5% by weight of a readily or sparingly soluble fatty acid salt; a salt of a fluorinated fatty acid, of an alkoxy-carboxylic acid, of a sulphoriamido carboxylic acid, of a fatty acid lactate, or of an alkylmalonic or alkylsuccinic acid; a sparingly soluble alkyl sulphonate; a sulphonated fatty acid alkyl ester; a fatty acid sulphonate; a fatty acid ester sulphonate; a perfluorinated alkyl sulphonate; a readily or sparingly soluble alkylbenzene sulphonate; a sulphated primary or secondary fatty alcohol; a soap, sulphated ester, amide, alkanolamide, mono- or polyglyceride or polyglycol ether, of a fatty alcohol or alkylphenol; a fatty acid ester with a mono- or poly-hydric alcohol; a fatty acid ester with an oligohydroxy compound or with a polyhydroxy compound; a polyethylene or polypropylene glycol ether having approximately from 2 to 23 ethylene glycol or ethylene oxide units of a fatty alcohol, of a fatty acid ester or of fatty amines derived from fatty alcohols; ethylene oxide or propylene oxide block copolymers having hydrophilic polyhydroxyethylene groups or hydrophobic poly hydroxypropykne groups having a molecular weight of from approximately 1000 to approximately 11000, the fatty acid each having from 6 to 34 carbon atoms and the fatty alcohol having approximately from 6 to 34 carbon atoms; as emulsifier, and present if the lipid phase is not self-emulsifying, and
(f) from approximately 0.5 to approximately 3% by weight of a synthetic gel-forming macromolecule, the units of which are vinyl alcohol, vinyl pyrrolidine, acrylic or methacrylic acid or their salts as gel structure former, and

27. A composition according to claim 26 wherein component (a) comprises isopropyl alcohol.

28. A composition according to claim 26 wherein component (b) comprises polyethylene glycol.

29. A composition according to claim 26 wherein component (d) comprises caprylicleapric acid esters of saturated fatty alcohols having from 12 up to and including 18 carbon atoms; or a silicone compound selected from dimethicone, dimethicone c opolyol, cyclome thic one, and mixtures thereof.

30. A composition according to claim 26 wherein component (e) comprises a polyethylene ether of a fatty alcohol.

31. A pharmaceutical composition according to claim 26 wherein component (f) comprises polyacrylic acid or a salt thereof.

32. A composition according to claim 26 wherein
component (a) comprises isopropyl alcohol:
component (h) comprises polyethylene glycol;
component (d) comprises caprylicicapric acid esters of saturated fatty alcohols having from 12 up to and including 18 carbon atoms; or a silicone compound selected from dimethicone, dimethicone copolyol, cyclomethicone, and mixtures thereof:
component (e) comprises a polyethylene ether of a fatty alcohol; and
component (t) comprises polyacrylie acid or a salt thereof.

33. A composition according to claim 32 wherein the at least one sensate agent is a physiological warming agent.

34. A composition according to claim 33 wherein the topically active non-steroidal anti-inflammatory agent is diclofenac or a pharmaceutically acceptable salt thereof and the at least one sensate agent is a vanilloid.

35. A composition according to claim 34 wherein the topically active non-steroidal anti-inflammatory agent is diclofcnac or a pharmaceutically acceptable salt thereof and the at least one sensate agent is vanillyl butyl ether.

36. A method for the treatment of painful conditions, inflammation and/or rheumatic diseases comprising administering topically to a warm-blooded animal an effective amount of a composition according to claim 1.

37. A method for the treatment of arthritic pain comprising administering topically to a warm-blooded animal an effective amount of a composition according to claim 15.

38. A method for the treatment of painful conditions, inflammation and/or rheumatic diseases comprising administering topically to a warm-blooded animal an effective amount of a composition according to claim 26.

39. A method for the treatment of arthritic pain comprising administering topically to a warm-blooded animal an effective amount of a composition according to claim 35.
